(19) United States
(12) Patent Application Publication
(10) Pub. No.: US 2015/0182213 A1
(43) Pub. Date: Jul. 2, 2015

(54) Collapsible Suture Anchor

(71) Applicant: Suspension Orthopaedic Solutions, LLC, Amoid, MD (US)

(72) Inventors: Jeffrey Gelfand, Severna Park, MD (US); Robert L. Richards, New Haven, CT (US); Hanspeter R. Bayer, Meriden, CT (US); Robert S. Collins, Severna Park, MD (US)

(73) Assignee: Suspension Orthopaedic Solutions, LLC, Arnold, MD (US)

(21) Appl. No.: 14/408,084

(22) PCT Filed: May 24, 2013
(86) PCT No.: PCT/US13/42589

§ 371 Date: Dec. 15, 2014

Related U.S. Application Data
(60) Provisional application No. 61/651,160, filed on May 24, 2012.

(51) Int. Cl. A61B 17/04

(52) U.S. Cl. CPC A61B 17/0401; A61B 2017/0414

(57) ABSTRACT

Described herein are devices and methods for securing sutures to tissue, particularly bone.

### CROSS-REFERENCE TO RELATED APPLICATION

This application claims priority to U.S. Provisional Application for Patent Ser. No. 61/651,160 filed on May 24, 2012, the entire disclosure of which is incorporated herein by reference.

### TECHNICAL FIELD

Described herein are devices and methods for securing sutures to tissue, particularly bone.

### BACKGROUND

As the treatment of injuries to joints and soft tissue has progressed in the orthopaedic medical arts, there has been a need for medical devices that can be used to attach tendons, ligaments and other soft tissue to bone or cartilaginous tissue. Further, the replacement of damaged soft tissues with implants has become common. Such restorations typically require the attachment the implant and/or soft tissues such as ligaments and tendons to bone.

One conventional orthopaedic procedure for reattaching soft tissue to bone is performed by initially drilling holes or tunnels at predetermined locations through a bone near a joint. Then, the surgeon approximates soft tissue to the surface of the bone using sutures threaded through these holes or tunnels. This method, although effective, is a time consuming procedure resulting in the generation of numerous bone tunnels. A known complication of drilling tunnels across bone is that nerves and other soft tissue structures may be injured by the drill bit or orthopaedic pin as it exits the far side of the bone. In addition, it is anatomically very difficult to reach and/or secure a suture/wire that has been passed through a tunnel. When securing the suture or wire on the far side of the bone, nerves and soft tissues can become entrapped and damaged.

In order to overcome some of the problems associated with the use of the conventional bone tunnel procedures, suture anchors have been developed and are frequently used to attach soft tissue to bone. A suture anchor is an orthopaedic, medical device that is typically implanted into a cavity drilled into a bone. Although less frequently, these devices have also been referred to as bone anchors. The cavity is typically referred to as a bore hole and usually does not extend through the bone. This type of bore hole is typically referred to as a “blind hole.” The bore hole is typically drilled through the outer cortex layer of the bone and into the inner cancellous layer. The suture anchor may be engaged in the bore hole by a variety of mechanisms including friction fit, barbs that are forced into the cancellous layer of bone, etc. Suture anchors are known to have many advantages including reduced bone trauma, simplified application procedures, and decreased likelihood of suture failure due to abrasion on bone. Suture anchors may be used in the Bankart shoulder reconstruction for repairing the glenohumeral ligament and may be used in surgical procedures such as rotator cuff repair and hip replacement. In addition, such anchors may be used in repair of tendon tears by direct attachment of bone-to-bone.

Suture anchors typically have at least one suture attached. This may be by means of a hole or opening for receiving the suture(s). At least one end and typically both ends of the suture strand extend out from the bore hole and are used to attach soft tissue. The suture anchors presently described in the art may be made of absorbable materials that absorb over time, or they may be made from various non-absorbable, biocompatible materials. Although most suture anchors described in the art are made from non-absorbable materials, the use of absorbable suture anchors may result in fewer complications since the suture anchor is absorbed and replaced by bone over time. In addition, the use of absorbable suture anchors may reduce the likelihood of damage to local joints caused by anchor migration.

Although suture anchors for attaching soft tissue to bone are available for use by the orthopaedic surgeon, there is a constant need for novel suture anchors having improved performance characteristics.

### SUMMARY

Described herein are devices and methods for securing sutures to tissue, particularly bone.

One embodiment described herein is a collapsible suture anchor for disposition in a tissue bore hole comprising: a plurality of jamming means comprising at least one internal channel therein; a cinching means interconnecting a plurality of jamming means; whereby when an operator tightens the cinching means, a plurality of jamming means are reoriented, and thereby secure the collapsible suture anchor to the tissue bore hole.

In some aspects described herein, the cinching means interconnects at least two jamming means via the internal channel.

In other aspects described herein, the cinching means forms a continuous loop encompassing a plurality of jamming means.

In other aspects described herein, the cinching means comprises a locking means.

In other aspects described herein, when an operator tightens the cinching means, the locking means traverses through at least one channel of the at least one jamming means, thereby locking the cinching means tight.

In other aspects described herein, at least one means for securing a suture is attached to the cinching means.

In other aspects described herein, at least one of the jamming means comprises an eyelet for securing a suture.

In other aspects described herein, the at least one suture is threaded through the eyelet of the at least one eyelet jamming means.

In other aspects described herein, the cinching means is affixed to at least one jamming means.

In other aspects described herein, the jamming means comprise one or more projections.

In other aspects described herein, the jamming means and cinching means are delivered using a delivery device.

In other aspects described herein, the delivery device is a flexible cannula.

In other aspects described herein, the delivery device is a wire or rod.

In other aspects described herein, the jamming means are spherical, oval, cubic, rectangular, triangular, trapezoidal, trefoil, quatrefoil, cinquefoil, hexafoil, heptafoil, octofoil, nonofoil, decafoil, multifoil, symmetric, asymmetric or various combinations thereof.

In other aspects described herein, the size and shape of the deployed collapsible suture anchor is smaller than the diameter than the tissue bore hole.

In other aspects described herein, the jamming means are medically compatible materials including stainless steel, titanium, nitinol, Vitalium, hydroxyapatite, calcium phosphate, polylactate (PLA), polyglycolate (PGA), poly lactide-co-glycolide copolymers (PLGA), polydioxanone (PDS), polyurethane, polyethylene, polypropylene, polystyrene, polytetrafluoroethylene, fluorinated ethylene propylene (FEP), perfluoroalkoxy (PFA), polyether ether ketone (PEEK), polyetherimide (PEI), polyamide imide (PAI), thermoplastic polyimides (TPI), polybenzimidazol (PBI), rubber, latex, silicone, other medically compatible metals, alloys, polymers, composites, and ceramics, porous variations thereof, or combinations thereof.

In other aspects described herein, the cinching means are medically compatible materials including suture, stainless steel, titanium, nitinol, Vitalium, polyethylene, polypropylene, polystyrene, polytetrafluoroethylene, fluorinated ethylene propylene (FEP), perfluoroalkoxy (PFA), polyether ether ketone (PEEK), polyetherimide (PEI), polyamide imide (PAI), thermoplastic polyimides (TPI), polybenzimidazol (PBI), rubber, latex, silicone, or other medically compatible metals, alloys, polymers, and composites, or combinations thereof.

In other aspects described herein, the tissue of disposition is fibrous tissue, connective tissue, ligament, tendon, cartilage, bone, or combinations thereof.

In other aspects described herein, the disposition of the collapsible suture anchor is within cortical bone; with cancellous bone; partially within cortical bone and partially within cancellous bone; on the exterior of bone on the side opposite insertion; on the exterior of bone on the side opposite insertion and partially within a cortical region; or various combinations thereof.

Another embodiment described herein is a collapsible suture anchor for disposition in a tissue bore hole comprising: a plurality of jamming means comprising at least one internal channel; a cinching means interconnecting a plurality of jamming means; a locking means; at least one suture connectively attached to at least one of the jamming means; whereby when an operator tightens the cinching means, the locking means traverses through at least one channel of the at least one jamming means, thereby locking the cinching means tight, thereby reorienting a plurality of jamming means and obstructing the tissue bore hole, thereby anchoring at least one suture to the tissue.

Another embodiment described herein collapsible suture anchor for disposition in a tissue bore hole comprising: a plurality of jamming means comprising at least one internal channel therein; wherein a plurality of the jamming means comprise one or more projections; a cinching means interconnecting a plurality of jamming means; whereby when an operator tightens the cinching means, a plurality of jamming means are reoriented, and thereby anchor the collapsible suture anchor to the tissue bore hole.

### BRIEF DESCRIPTION OF THE DRAWINGS

FIG. 1 illustrates an exemplary embodiment of a collapsible suture anchor as described herein. FIG. 1A shows the collapsible suture anchor after deployment into a tissue bore hole. FIG. 1B shows the collapsible suture anchor after collapsing. FIG. 1C shows the collapsed anchor.

FIG. 2 illustrates exemplary embodiments of an eyelet jamming means and a locking means as described herein. FIG. 2A shows front, side, and section views, respectively of an exemplary embodiment of an eyelet jamming means. FIG. 2B shows an exemplary locking means and the progression of a locking means through an eyelet jamming means. FIG. 2C shows a blowup of an exemplary locking means and a suture.

FIG. 3 illustrates another exemplary embodiment of a collapsible suture anchor as described herein. FIG. 3A shows an exemplary collapsible suture anchor after deployment into a tissue bore hole. In this embodiment, an eyelet jamming means is placed in between two jamming means on each side. A cinching suture connects all of the jamming means. Upon cinching and locking, the eyelet jamming means exits the tissue bore leaving the four jamming means within the tissue bore hole, thereby anchoring a suture through the eyelet jamming means.

FIG. 4 illustrates yet another exemplary embodiment of a collapsible suture anchor as described herein. FIG. 4A shows an exemplary collapsible suture anchor after deployment into a tissue bore hole. In this embodiment, the four jamming means are triangular, wing shaped plates that come together when cinched to form a pyramidal quatrefoil with the eyelet jamming means in the center. FIG. 4B shows a side view of the collapsed suture anchor with sutures attached to the eyelet jamming means traversing through the tissue bore hole. FIG. 4C shows an axial view of the collapsed suture anchor, illustrating the pyramidal quatrefoil shape formed by the jamming means and eyelet jamming means upon cinching.

FIG. 5 illustrates still another exemplary embodiment of a collapsible suture anchor as described herein. FIG. 5A shows an exemplary collapsible suture anchor after deployment into a tissue bore hole. In this embodiment, a plurality of jamming means, including the eyelet jamming means, are non-spherical plates that come together when cinched to form a cinquefoil or rosette assembly. FIG. 4B shows a side view of the collapsed suture anchor with sutures attached to the eyelet jamming means traversing through the tissue bore hole.

FIG. 6 illustrates another exemplary embodiment of a collapsible suture anchor as described herein. FIG. 6A shows an exemplary collapsible suture anchor after deployment into a tissue bore hole. In this embodiment, plurality of spherical jamming means are interconnected and connected to a non-spherical eyelet jamming means. FIG. 6A also shows an alternative means for routing the cinching means through the collapsable suture anchor. FIG. 6B shows a side view of the collapsed suture anchor.

FIG. 7 illustrates yet another exemplary embodiment of a collapsible suture anchor as described herein. FIG. 7A shows an exemplary collapsible suture anchor after deployment into a tissue bore hole. In this embodiment, the jamming means are wedge-shaped plates that have pointed projections that can dig-in or bite into the sides of the tissue bore hole. FIG. 7B shows the collapsed suture anchor after cinching where the wedge-shaped jamming means have articulated and the projections have become embedded in the tissue bore hole walls.

### DETAILED DESCRIPTION

Described herein are devices and methods for securing sutures to tissue, particularly bone. When performing surgery or implantation of medical implants it is sometimes necessary to anchor sutures in rigid tissue such as cartilage or bone. Accordingly, there is a need for a means of anchoring sutures to tissue in a rapid and minimally invasive manner.

One embodiment described herein is a collapsible suture anchor for disposition in a tissue bore hole comprising: a plurality of jamming means comprising at least one internal channel therein; a cinching means interconnecting a plurality of jamming means; whereby when an operator tightens the cinching means, a plurality of jamming means are reoriented, and thereby anchor the collapsible suture anchor to the tissue bore hole.

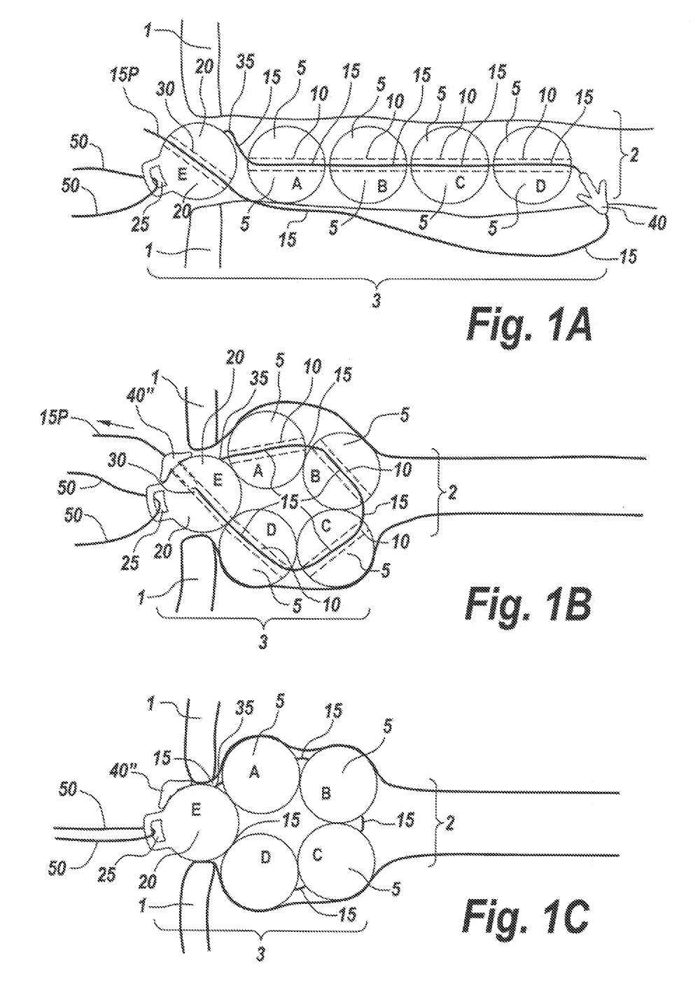

Referring to FIG. 1, an exemplary illustration of one embodiment of the collapsible suture anchor described herein is shown. FIG. 1A shows the collapsible suture anchor after deployment into a tissue 1 bore hole 2. The collapsible suture anchor 3 comprises a plurality of jamming means 5 (A, B, C, D); at least one cinching means 15; at least one locking means 40; at least one eyelet jamming means 20 (E); and at least one suture 50. The plurality of jamming means 5 can have a channel 10 traversing through their respective bodies. Each jamming means 5 is interconnected by at least one cinching means 15 that passes through each of the respective channels 10 of the jamming means 5. At least one locking means 40 is connected to the cinching means 15. The cinching means 15 comprises a ligature such as a suture or wire, for example. At least one jamming means is an eyelet jamming means 20 that comprises a channel 30 similar to that of the jamming means 5. The eyelet jamming means 20 (E) differs from the jamming means 5 (A, B, C, D) in that it comprises an eyelet 25 or orifice for threading at least one suture 50. In some embodiments, the cinching means 15 is attached to 35 the eyelet jamming means body 20 (E).

In FIGS. 1B and 1C, the collapsible suture anchor 3 is illustrated after “collapse” i.e., after the jamming means have been cinched and locked by the operator. During cinching, the operator pulls the proximal end of the cinching means 15 (P). Upon pulling the proximal end of the cinching means 15 (P), the at least one locking means 40 is pulled proximally and through the interior channel 30 of the eyelet jamming means 20 (E). The locking means 40 is capable of deforming to traverse the eyelet jamming means internal channel 30, but once through, cannot re traverse back through the channel. The locking means “locks” the jamming means 5 and eyelet jamming means 20 (E) in the cinched conformation. This cinching and locking serves to transform the collapsible suture anchor 3 from a flexible, linear “beads on a string” conformation (FIG. 1A) to a bunched, semi-rigid assembly that jams in the tissue bore hole orifice 2 (O) (FIG. 1C) and serves as an anchor for one or more sutures 50. After cinching the cinching means 15 ligature and locking the locking means 40, the proximal tag end of the cinching means 15 (P) may be cut and removed (see FIG. 1C).

The shape of the jamming means 20 (E) and the routing of the cinching means 15 determines the overall shape of the collapsible suture anchor 3 in its collapsed state. In some aspects, the overall size and/or shape of the collapsible suture anchor in the deployed state is smaller than the diameter of the tissue bore, so that delivery, collapse, and reorientation of the jamming means is possible. In the illustration in FIG. 1B, the exemplary spherical jamming means 5 (A, B, C, D) and eyelet jamming means 20 (E) form a planar pentagonal shape, with the eyelet jamming means 20 and suture 50 protruding through the tissue bore hole orifice 2 (O). FIG. 1C shows another view of the collapsed suture anchor (i.e., after cinching and locking) where the interior channels 10, 30 of the jamming means 5 (A, B, C, D) and eyelet jamming means 20 (E) are not shown. The suture 50 can then be used by an operator for suturing as needed.

Multiple anchoring modes and sites are contemplated by the collapsible suture anchor described herein. In some aspects, the collapsable suture anchor described herein may be deployed wholly within a tissue bore hole. The bore hole may be made by an operator using tools and methods known to those having ordinary skill in the art. In some aspects, the collapsible suture anchor may be deployed within a bore hole in firm tissue, cartilage, or bone. In other aspects, the collapsible suture anchor may be deployed within a cortical region of bone. In other aspects, the collapsible suture anchor may be deployed within a cancellous region of bone. In other aspects, the collapsible suture anchor may be deployed partially within a cancellous region and partially within a cortical region. In other aspects, the collapsible suture anchor may be deployed on the exterior of bone on the side opposite insertion. In other aspects, the collapsible suture anchor may be deployed partially on the exterior of bone on the side opposite insertion and partially within a cortical region.

The jamming means 5 and/or eyelet jamming means 20 can comprise a variety of medically compatible materials including stainless steel, titanium, nitinol, Vitalium, hydroxyapatite, calcium phosphate, polylactate (PLA), polyglycolate (PGA), poly lactide-co-glycolide copolymers (PLGA), polydioxanone (PDS), silicone, polyurethane, polyethylene, polypropylene, polystyrene, polytetrafluoroethylene, fluorinated ethylene propylene (FEP), perfluoroalkoxy (PFA), polyether ether ketone (PEEK), polyetherimide (PEI), polyamide imide (PAI), thermoplastic polyimides (TPI), polybenzimidazol (PBI), rubber, latex, or other medically compatible metals, alloys, polymers, composites, or ceramics as understood by those skilled in the art. The jamming means 5 or eyelet jamming means 20 may be solid or porous. The jamming means 5 or eyelet jamming means 20 may be deformable or rigid; they may be inert or absorbable, or they may effuse growth factors, antibiotics, drugs, or the like.

The cinching means generally comprises a ligature, such as a suture or a wire, that is used to join the jamming means together and/or collapse the collapsible suture anchor. The cinching means can comprise a variety of materials such as medically compatible stainless steel, titanium, nitinol, suture, plastic extrusions, rubber, latex, silicon, or other medically compatible metals, alloys, polymers, plastics, or composites, as understood by those skilled in the art. In some aspects, at least one end of the cinching means is affixed to one or more jamming means or eyelet jamming means. In other aspects, a plurality of ends of cinching means is affixed to one ore more jamming means or eyelet jamming means. In some aspects, the cinching means forms a continuous loop and joins a plurality of the jamming means together by being threaded through their respective internal channels. In some aspects, the cinching means is pulled by the operator to collapse the collapsible suture anchor. In some aspects, both ends of the cinching means are pulled by the operator to collapse the collapsible suture anchor. In other aspects, the cinching means joins a plurality of the jamming means together. In some aspects, there is at least one cinching means. In other aspects, there are multiple cinching means, which may or may not be interconnected.

In some aspects, the operator pulls the suture to collapse the collapsible suture anchor and the cinching means joins a plurality of the jamming means together. The routing of the cinching means circuitously through the jamming means, eyelet jamming means, and/or locking means may be in any order or sequence to effectively deliver, deploy, collapse, orient, and secure the suture anchor in a tissue bore. Examples described herein are merely for illustration purposes and multiple effective routings of the cinching means are envisioned for delivering, deploying, collapsing, orienting, and securing the collapsible suture anchors described herein.

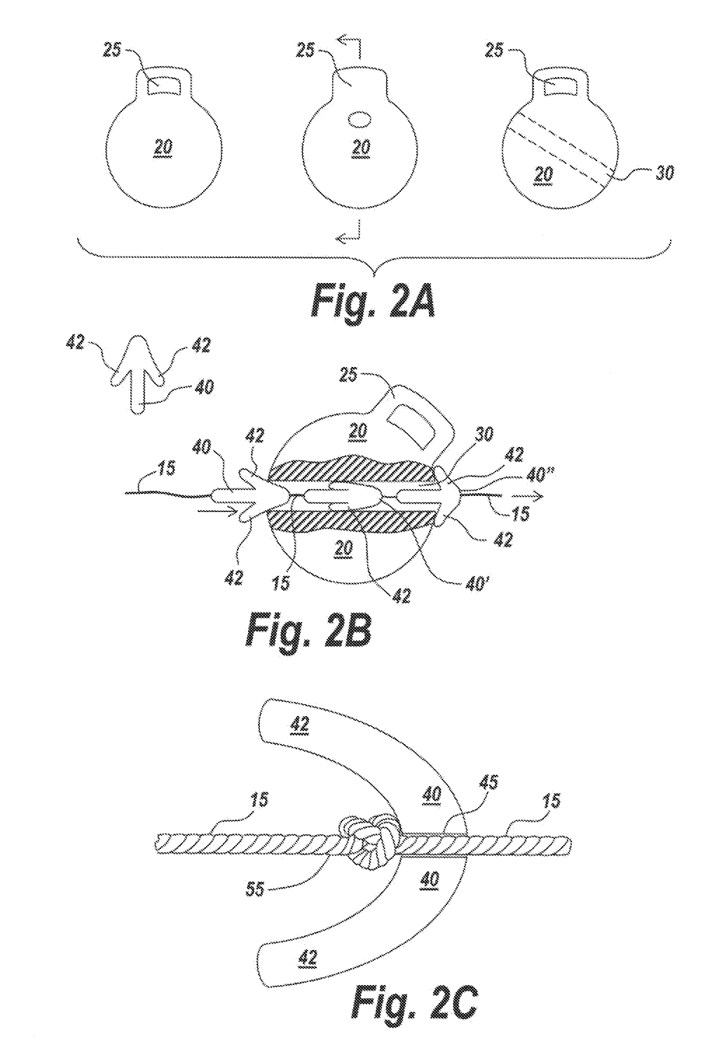

In FIG. 2, exemplary embodiments of the eyelet jamming means 20 and the locking means 40 as described herein are illustrated. The eyelet jamming means 20 is shown in FIG. 2A in back and side views respectively. The third panel of FIG. 2A, shows a schematic of an exemplary channel 30 traversing the body of the eyelet jamming means 20. In this example, the eyelet 25 is an extension of the spherical body 20, with an orifice traversing two of the sides to form an eyelet opening 25. In some embodiments, the eyelet 25 can be a separate component attached to a 5 jamming means. In some embodiments, the channel can be straight. In other embodiments, the channel can be curved or at an angle. The orifice or opening in the eyelet may be any shape including round, oval, semicircle, square, rectangular, triangular, trapezoidal, or polygonal. The eyelet may additionally contain a notch or catch for temporarily or permanently securing at least one suture 50 and preventing it from freely moving.

An exemplary embodiment of a locking means 40 as described herein is illustrated in FIG. 2B. The locking means 40 comprises an object that may pass through at least the internal channel 30 of the eyelet jamming means 20 in one direction, but cannot pass back through the channel in the opposite direction. In some aspects, the locking mechanism can also traverse the internal channels 10 of the jamming means 5. In some aspects, multiple locking means may be utilized. An exemplary embodiment of the locking means 40 is an arrowhead shaped object that has “barbs” 42 that are deformable and capable of bending or flexing during transversal of the internal channel 30 of the eyelet jamming means 20. The process is illustrated in panel FIG. 2B. The arrowhead-shaped locking mechanism is affixed on a cinching means ligature 15 on the distal (left side of FIG. 2D) of the eyelet jamming means 20. As the cinching means 15 is pulled proximally (to the right in FIG. 2D), the channel 30 of eyelet jamming means 20 compresses the barbs 42 of the locking means rearward 40′ (opposite the direction of pulling), allowing the locking means to slide through the channel 30. Once the locking means 40 is pulled proximally (right in FIG. 2B) through the eyelet jamming means 20 channel 30, the locking means 40 barbs 42 re-expand and prevent the locking means 40″ from backsliding or being pulled back from the distal direction (to the left in FIG. 2B). An exemplary embodiment of a locking means 40 is illustrated in FIG. 2C. In this example, the barbs 42 are exaggerated for clarity. The locking means 40 comprises a channel 45 that permits the cinching means ligature 15 to traverse through the locking means 40. The locking means 40 is affixed to the cinching means ligature 15 through a securing means 55. In some embodiments, the securing means 55 can be a knot in the cinching means 15.

Like the jamming means 5, eyelet jamming means 20, or cinching means 15, the locking means 40 can comprise a variety of medically compatible materials including, but not limited to, plastics, metal, or composites. The material should be flexible enough so that the locking means barbs 42 are not damaged or permanently bent during transversal through a jamming means channel. In some embodiments, the locking means can have multiple barbs. In other embodiments, the locking means can comprise a plurality of individual locking means objects.

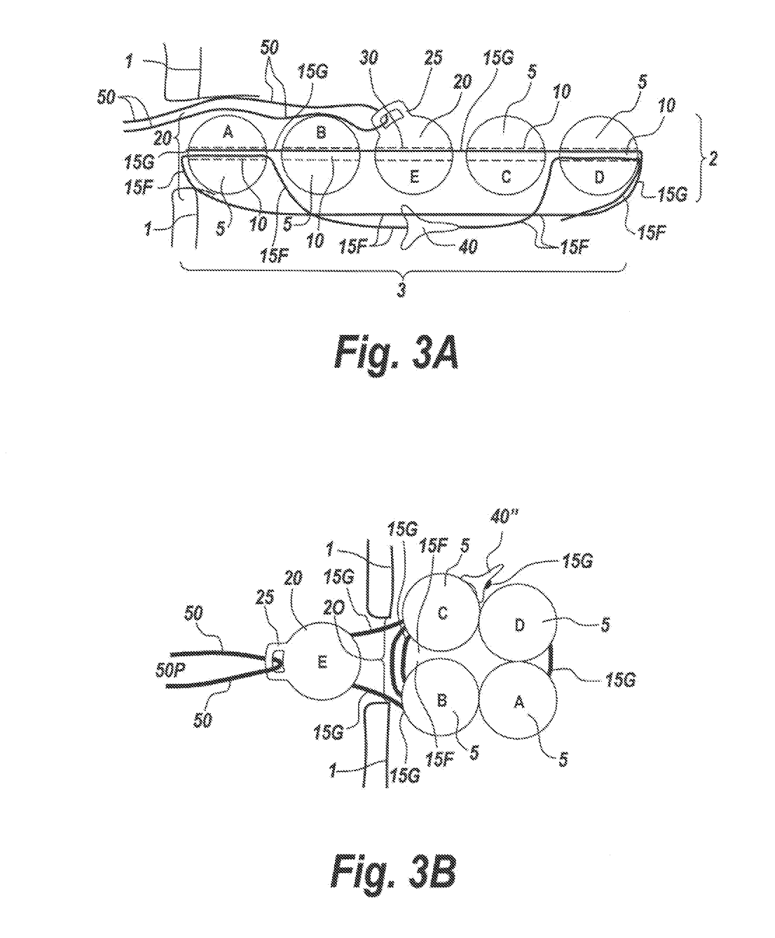

An alternative embodiment of a collapsible suture anchor as described herein is illustrated in FIG. 3. This exemplary embodiment comprises four jamming means 5 (A, B, C, D), one eyelet jamming means 20 (E), a locking means 40, a cinching means 15, and at least one suture 50 , that are inserted into a tissue 1 bore hole 2. This embodiment differs from that shown in FIG. 1 in that one of the cinching means 15 (F) is not attached to the eyelet jamming means 20 (E), but rather forms an continuous loop around jamming means 5 A and D. A second, self-contained cinching means 15 (G) loop interconnects all of the jamming means 5 (A, B, C, D) and the eyelet jamming means 20 (E). When an operator pulls the proximal ends of the suture 50 (P), the eyelet jamming means 20 (E) is pulled through the tissue bore hole, leaving the other jamming means 5 (A, B, C, D) on the interior of the tissue 1 bore hole 2. As the eyelet jamming means 20 (E) is further pulled out of the tissue bore hole, the jamming means 5 (A, B, C, D) are bunched together and jam the tissue bore hole orifice 2 (O). The jamming means 5 (A, B, C, D) are prevented from linearizing and sliding through the tissue bore hole owing to the cinching means 15 (G) ligature, which runs through the internal channel 10 of jamming means 5 (A, B, C, D) and the internal channel 30 of the eyelet jamming means 20 (E). Further, a self-contained cinching means loop 15 (G) secures jamming means 5 (A) and 5 (D) adjacent to each other, further preventing the jamming means complex 5 (A, B, C, D) from linearizing and sliding through the tissue bore hole orifice 2 (O). In this exemplary embodiment, the cinching means loops 15 (G) and 15 (F) are self-contained and do not need to be pulled and/or cut by the operator. The orientation and potential interconnectivity of cinching means 15 (G) and 15 (F) may be in any manner necessary to facilitate jamming of the tissue bore hole orifice 2 (O) and is not limited to the embodiments shown in FIG. 3A and 3B.

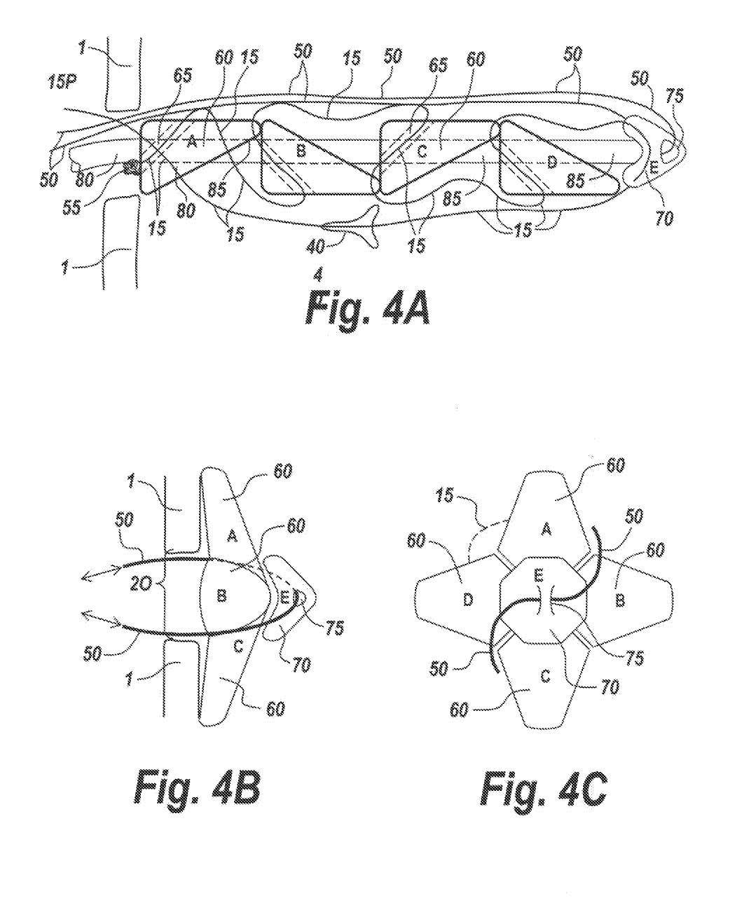

Yet another exemplary embodiment of the collapsible suture anchor as described herein is illustrated in FIG. 4. In this embodiment, the jamming means are planar, wing-shaped, wedges 60 (A, B, C, D) and the eyelet jamming means 70 (E) has a pyramidal shape. When cinched to form the functional jamming unit, the jamming means 60 (A, B, C, D) and the eyelet jamming means 70 (E) form a quatrefoil pyramid with the four jamming means 60 (A, B, C, D) forming the points and base of the pyramid and the eyelet jamming means 70 (E) forming the capstone of the pyramid. See FIG. 4 C.

In this exemplary embodiment, the jamming means 60 (A, B, C, D) and the eyelet jamming means 70 (E) are inserted into the tissue bore hole 2 aligned on a nitinol wire 80, for example (FIG. 4A). After deployment into a tissue bore hole 2, the nitinol delivery wire 80 is removed by the operator. Further, in this example, the jamming means 60 (A, B, C, D) have two internal channels; one channel serves as a conduit for the cinching means 15 and the other is an axial channel (not visible in the drawings) for traversal of the nitinol 80 wire during deployment. The cinching means 15 is anchored 55 to the terminal jamming means 60 (A); in this example the attachment is a knot that prevents the cinching means 15 from being pulled through the internal channel of jamming means 60 (A). The anchor 55 may be any means for connection of the cinching means 15 to the jamming means 60 (A). The cinching means 15 circuitously traverses through the respective internal channels of jamming means 60 (A), 60 (B), 60 (C), 60 (D), and eyelet jamming means 70 (E). After exiting the internal channel of eyelet jamming means 70 (E), the cinching means 15 traverses through the internal channel of locking means 40 and then through a third internal channel 80 in jamming means 60 (A). The proximal end of the cinching means 15 (P) passes through the tissue bore hole orifice 2 (O). See FIG. 4A. When an operator pulls the proximal end of the cinching means 15 (P), the locking means 40 is pulled proximally through the third internal channel 80 in jamming means 60 (A) and locks. Concomitantly, the cinching means 15 pulls together jamming means 60 (A), 60 (B), 60 (C), 60 (D), and eyelet jamming means 70 (E) and facilitates assembly of the pyramidal suture anchor, which obstructs the suture anchor from exiting the tissue bore hole orifice 2 (O). See FIG. 4B and 4C. The operator then can cut the proximal end of the cinching means 15 (P) adjacent to the locking means 40 and locked jamming means 60 (A). One or more sutures 50 traverse through the eyelet 75 of eyelet jamming means 70 (E) and then extend around the sides of the assembled suture anchor and then proximally 50 (P) out the tissue bore hole orifice 2 (O) (FIG. 4B and 4C). The operator may then use the sutures 50 for suturing as needed.

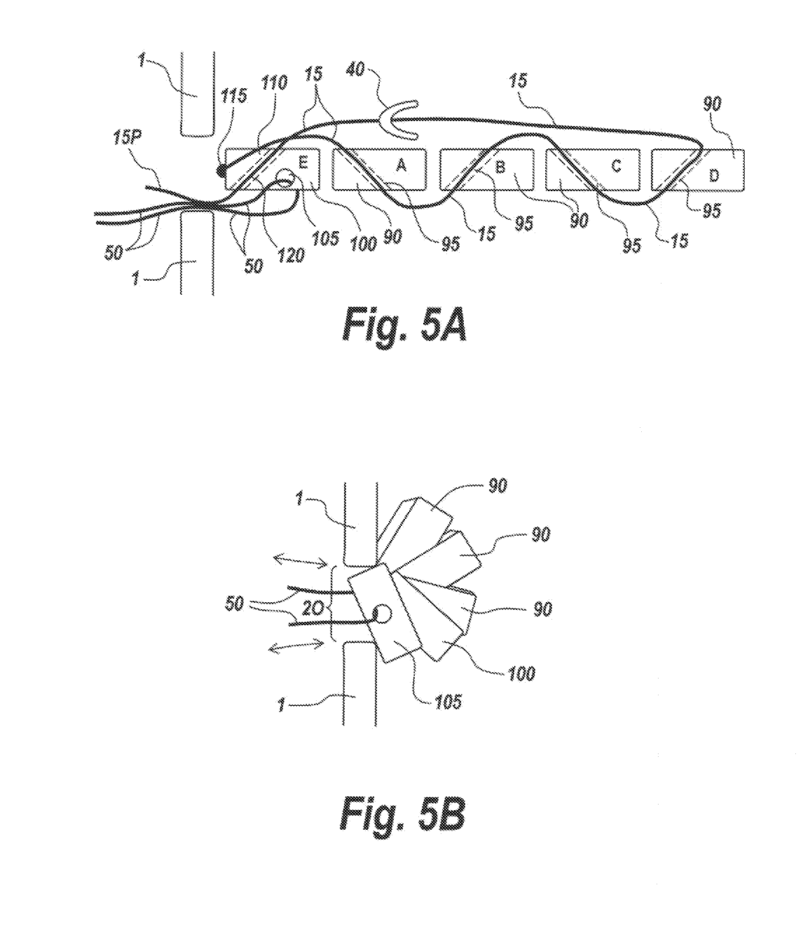

Still yet, another exemplary embodiment of a collapsible suture anchor as described herein is illustrated in FIG. 5. In this embodiment, the collapsible suture anchor comprises four jamming means 90 (A, B, C, D) and one eyelet jamming means 100 (E) comprising rectangular plates. The jamming means 90 (A, B, C, D) and eyelet jamming means 100 (E) are interconnected by a cinching means 15 that is anchored 115 to the proximal end of the eyelet jamming means 100 (E). The anchor 115 may be a knot that prevents the cinching means 15 from passing through one internal channel of jamming means 100 (E) or may be any other means for anchoring the cinching means 15 to the jamming means 100 (E). The cinching means 15 circuitously traverses through the respective internal channels of jamming means 90 (A), 60 (B), 60 (C), and 60 (D). After exiting the internal channel 95 of eyelet jamming means 90 (D), the cinching means 15 traverses through the internal channel of locking means 40 and then through a third internal channel 120 in eyelet jamming means 100 (E). The proximal end of the cinching means 15 (P) passes through the tissue bore hole orifice 2 (O). See FIG. 5A. When an operator pulls the proximal end of the cinching means 15 (P), the locking means 40 is pulled proximally through a third internal channel 120 of eyelet jamming means 100 (E) and locks. Concomitantly, the cinching means 15 pulls together jamming means 60 (A), 60 (B), 60 (C), 60 (D), and eyelet jamming means 100 (E), and facilitates assembly of a cinquefoil or rosette-shaped suture anchor. The collapsed suture anchor obstructs the tissue bore hole orifice 2 (O) and secures at least on suture 50 to the tissue. See FIG. 5B. The operator then can cut the proximal end of the cinching means 15 (P) adjacent to the locking means 40 and locked eyelet jamming means 100 (E). One or more sutures 50 traverse through the eyelet 105 of eyelet jamming means 100 (E) and then extends proximally 50 (P) out the orifice of the tissue bore hole 2 (O). See FIG. 5B. The operator may then use the sutures 50 for suturing as needed.

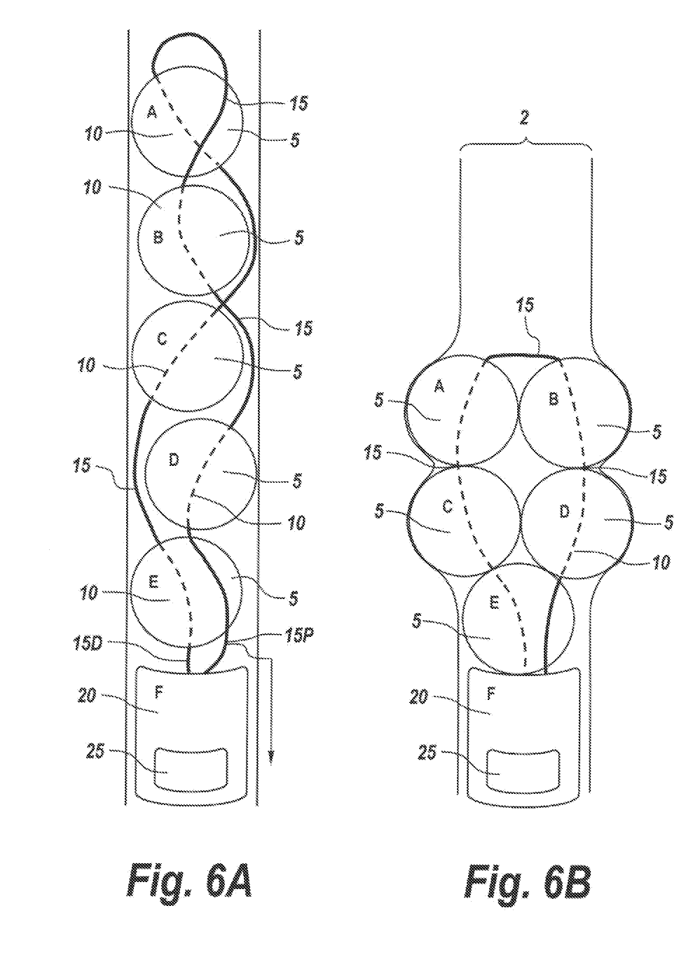

Another exemplary embodiment of a collapsible suture anchor as described herein is illustrated in FIG. 6. FIG. 6A shows the collapsible suture anchor after deployment into a tissue bore hole 2. FIG. 6 also illustrates an alternative routing of the cinching means. Cf. FIG. 1 and FIG. 3. The collapsible suture anchor comprises a plurality of jamming means 5 (A, B, C, D, E); at least one cinching means 15; and at least one eyelet jamming means 20 (F). The plurality of jamming means 5 (A, B, C, D, E) have a channel 10 traversing through their respective bodies. The channel is depicted in the drawing by the shaded appearance of the cinching means in the interior of the jamming means 5 (A, B, C, D, E). Each jamming means 5 (A, B, C, D, E) is interconnected by at least one cinching means 15 that passes through each of the respective channels 10 of the jamming means 5 (A, B, C, D, E). At least one jamming means is an eyelet jamming means 20 that can comprises a channel similar to that of the jamming means 5. The eyelet jamming means 20 (F) differs from the jamming means 5 (A, B, C, D, E) in that it comprises an eyelet 25 or orifice for threading at least one suture (not shown). The cinching means 15 comprises a ligature such as a suture or wire that can comprise a variety of medically compatible materials, as described herein. In this exemplary embodiment, the cinching means 15 traverses through the eyelet jamming means 20 (F), and the traverses through jamming means 5 E, C, A, B, D (i.e., in this order) and then re-traverses the eyelet jamming means 20 (F). The routing of the cinching means circuitously through the jamming means may be accomplished in many different manners so as to orient the jamming means in particular positions or shapes. The cinching means 15 may be attached to the eyelet jamming means 20 (F) at one or both traversal points or the ends may be secured by the operator by knotting or other means. In some embodiments, the eyelet jamming means 20 (F) may have at attachment means for securing one or more cinching means 15 and/or sutures. In addition, in this exemplary embodiment, no locking means is utilized. In this example, the jamming means 5 (A, B, C, D, E) are spherical and are slightly smaller than the diameter of the tissue bore hole 2. This permits the jamming means to reorient upon collapse and cinching. The collapsible suture anchor can be delivered to the tissue bore hole via a flexible cannula, for example. The jamming means may have smooth surfaces or may have a plurality of projections or barbs that facilitate interaction with the interior walls of the tissue bore hole 2. In some aspects, the entire surface of the jamming means may be roughened, sintered, or porous to facilitate adhesion to the tissue bore wall 2 and/or encourage tissue-ingrowth into the jamming means.

FIG. 6B illustrates the collapsible suture anchor shown in FIG. 6A after collapse. In this aspect, an operator has pulled at least one end of the cinching means 15, causing the jamming means 5 (A, B, C, D, E) to collapse, i.e., re-arrange their orientations, and bind with the tissue bore walls 2, thereby anchoring the collapsible suture anchor to the tissue bore 2. The collapsible suture anchor is anchored by the friction and resistance caused by the jamming means 5 (A, B, C, D, E) against the tissue bore walls 2, and because the collapsed or rearranged shape of the jamming means 5 (A, B, C, D, E) is larger than the diameter of the tissue bore orifice. If the jamming means 5 (A, B, C, D, E) comprise projections or burrs, these further prevent slippage of the collapsed suture anchor by binding to the interior of the tissue bore hole walls.

In some embodiments, the cinching means 15 is attached to the eyelet jamming means body 20 (F). In this exemplary embodiment a locking means, as described herein, is not used. The operator can tie together the tag ends of the cinching means 15. Alternatively, if the distal end of the cinching means 15 D is affixed to the eyelet jamming means 20 (F), then the operator can knot the proximal end of the cinching means 15 P adjacent to where it exits from an internal channel (not shown) in the eyelet jamming means 20 (F) to secure it from sliding back through the channel and lock the cinching means in place. In some embodiments, the eyelet jamming means 20 (F) can have a securing means for securing at least one end of the cinching means. In some aspects, the securing means can comprise a unidirectional notch or grove adjacent to internal channel where the cinched cinching means can be pulled into and permanently or temporarily secured.

The collapsible suture anchor can be removed by cutting and/or relieving the tension on the cinching means. The operator can pull out the cinching means, disarticulating the plurality of jamming means and then individually remove the eyelet jamming means and plurality of jamming means using instruments known by those having skill in the art.

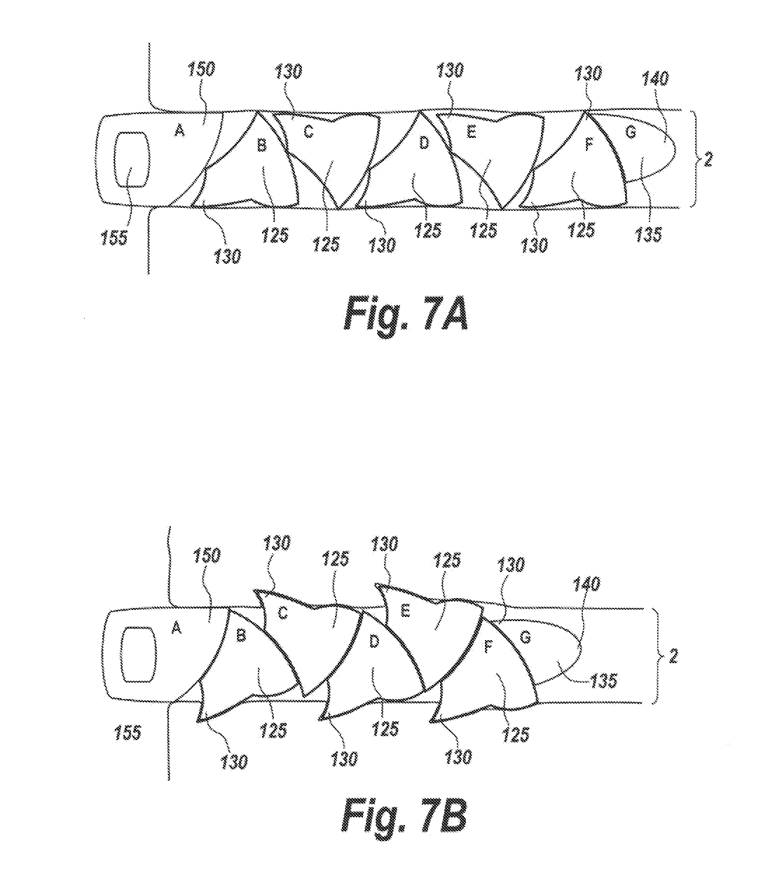

Yet another exemplary embodiment of a collapsible suture anchor as described herein is illustrated in FIG. 7. In this embodiment, the collapsible suture anchor comprises five jamming means 125 (B, C, D, E, F) that are triangular or wedge-shaped plates that have pointed projections 130 that can dig-in or bite into the sides of the tissue bore hole 2. The primary jamming means 135 has a pointed tip 140 and a pointed projection 130. The pointed tip 140 permits penetration through tissue. The tip 140 may have one or more barbs (not shown) for securing the primary jamming means to the bottom of the tissue bore hole 2. The primary jamming means 135 projection 130 is analogous to that of the wedge-shaped jamming means 125 (B, C, D, E, F) and is useful for biting into the tissue bore walls and prevents back slippage of the collapsible suture anchor. The eyelet jamming means 150 extends out of the tissue bore hole and comprises at least one eyelet 155 for securing one or more sutures (not shown). In this aspect, the eyelet jamming means 150, five wedge-shaped jamming means 125 (B, C, D, E, F), and the primary jamming means 135 can be interconnected by at least one cinching means (not shown in the figure). The at least one cinching means comprises a ligature such as a suture or wire that can comprise a variety of medically compatible materials, as described herein. The cinching means may be affixed to one or more of the jamming means 125 (B, C, D, E, F), eyelet jamming means 150, or primary jamming means 135, and may traverse among or in between such jamming means via internal channels as described herein. When an operator cinches the cinching means, the jamming means are drawn together. Owing to the geometry of the triangular wedge-shaped jamming means 125 (B, C, D, E, F), the projecting points 130 are forced outward, and bind into the tissue bore hole walls 2. The cinching means can be pulled, tensioned, and secured using any of the means described herein.

FIG. 7B shows the collapsed suture anchor after cinching where the wedge-shaped jamming 125 (B, C, D, E, F) means have articulated and slid against each other and the projections 130 have become embedded in the tissue bore hole walls 2. After cinching, the collapsible suture anchor is secured in the tissue bore. The collapsible suture anchor can be removed by relieving the tension of the cinching mean, slightly pushing the collapsible suture anchor toward the bottom of the tissue bore to disengage the projecting points 130 from the tissue bore walls, and then applying slight tension to one or more of the cinching means to retract the projecting points so that they become flush with the adjacent jamming means. The collapsible suture anchor can then be removed by the operator by pulling on the eyelet jamming means 150 in a direction opposite that of the tissue bore hole 2.

The scope of the devices and methods described herein includes all combinations of embodiments, aspects, examples, steps, and preferences herein described.

What is claimed is:

1. A collapsible suture anchor for disposition in a tissue bore hole comprising:
a plurality of jamming means comprising at least one internal channel therein;
a cinching means interconnecting a plurality of jamming means;
whereby when an operator tightens the cinching means, a plurality of jamming means are reoriented, and thereby secure the collapsible suture anchor to the tissue bore hole.

2. The collapsible suture anchor of claim 1, wherein the cinching means interconnects at least two jamming means via the internal channel.

3. The collapsible suture anchor of claim 1, wherein the cinching means forms a continuous loop encompassing a plurality of jamming means.

4. The collapsible suture anchor of claim 1, wherein the cinching means comprises a locking means.

5. The collapsible suture anchor of claim 4, whereby when an operator tightens the cinching means, the locking means traverses through at least one channel of the at least one jamming means, thereby locking the cinching means tight.

6. The collapsible suture anchor of claim 1, wherein at least one means for securing a suture is attached to the cinching means.

7. The collapsible suture anchor of claim 1, wherein at least one of the jamming means comprises an eyelet for securing a suture.

8. The collapsible suture anchor of claim 7, wherein the at least one suture is threaded through the eyelet of the at least one eyelet jamming means.

9. The collapsible suture anchor of claim 1, wherein the cinching means is affixed to at least one jamming means.

10. The collapsible suture anchor of claim 1, wherein the jamming means comprise one or more projections.

11. The collapsible suture anchor of claim 1, wherein the jamming means and cinching means are delivered using a delivery device.

12. The collapsible suture anchor of claim 11, wherein the delivery device is a flexible cannula.

13. The collapsible suture anchor of claim 11, wherein the delivery device is a wire or rod.

14. The collapsible suture anchor of claim 1, wherein the jamming means are spherical, oval, cubic, rectangular, triangular, trapezoidal, trefoil, quatrefoil, cinquefoil, hexafoil, heptafoil, octofoil, nonofoil, decafoil, multifoil, symmetric, asymmetric or various combinations thereof.

15. The collapsible suture anchor of claim 1, wherein the size and shape of the deployed collapsible suture anchor is smaller than the diameter than the tissue bore hole.

16. The collapsible suture anchor of claim 1, wherein the jamming means are medically compatible materials including stainless steel, titanium, nitinol, Vitalium, hydroxyapatite, calcium phosphate, polylactate (PLA), polyglycolate (PGA), poly lactide-co-glycolide copolymers (PLGA), polydioxanone (PDS), polyurethane, polyethylene, polypropylene, polystyrene, polytetrafluoroethylene, fluorinated ethylene propylene (FEP), perfluoroalkoxy (PFA), polyether ether ketone (PEEK), polyetherimide (PEI), polyamide imide (PAI), thermoplastic polyimides (TPI), polybenzimidazol (PBI), rubber, latex, silicone, other medically compatible metals, alloys, polymers, composites, and ceramics, porous variations thereof, or combinations thereof.

17. The collapsible suture anchor of claim 1, wherein the cinching means are medically compatible materials including suture, stainless steel, titanium, nitinol, Vitalium, polyethylene, polypropylene, polystyrene, polytetrafluoroethylene, fluorinated ethylene propylene (FEP), perfluoroalkoxy (PFA), polyether ether ketone (PEEK), polyetherimide (PEI), polyamide imide (PAI), thermoplastic polyimides (TPI), polybenzimidazol (PBI), rubber, latex, silicone, or other medically compatible metals, alloys, polymers, and composites, or combinations thereof.

18. The collapsible suture anchor of claim 1, wherein the tissue of disposition is fibrous tissue, connective tissue, ligament, tendon, cartilage, bone, or combinations thereof.

19. The collapsible suture anchor of claim 1, wherein the disposition of the collapsible suture anchor is within cortical bone; with cancellous bone; partially within cortical bone and partially within cancellous bone; on the exterior of bone on the side opposite insertion; on the exterior of bone on the side opposite insertion and partially within a cortical region; or various combinations thereof.

20. A collapsible suture anchor for disposition in a tissue bore hole comprising:
a plurality of jamming means comprising at least one internal channel;
a cinching means interconnecting a plurality of jamming means;
a locking means;
at least one suture connectively attached to at least one of the jamming means;
whereby when an operator tightens the cinching means, the locking means traverses through at least one channel of the at least one jamming means, thereby locking the cinching means tight, thereby reorienting a plurality of jamming means and obstructing the tissue bore hole, thereby anchoring at least one suture to the tissue.

21. A collapsible suture anchor for disposition in a tissue bore hole comprising:
a plurality of jamming means comprising at least one internal channel therein;
wherein a plurality of the jamming means comprise one or more projections;
a cinching means interconnecting a plurality of jamming means;
whereby when an operator tightens the cinching means, a plurality of jamming means are reoriented, and thereby anchor the collapsible suture anchor to the tissue bore hole.
